(19) United States
(12) Patent Application Publication
(10) Pub. No.: US 2017/0128556 A1
(43) Pub. Date: May 11, 2017

(54) IMMUNOTHERAPY USING T PRECURSOR CELLS DERIVED FROM PLURIPOTENT STEM CELLS HAVING REARRANGED T CELL RECEPTOR GENES

(71) Applicant: THYAS CO. LTD., Kyoto (JP)

(72) Inventors: Hiroshi Kawamoto, Kyoto (JP); Kyoto Masuda, Kyoto (JP)

(21) Appl. No.: 15/108,207

(22) PCT Filed: Dec. 26, 2014
(86) PCT No.: PCT/JP2014/084546

§ 371 Date: Dec. 28, 2016

Related U.S. Application Data
(60) Provisional application No. 61/920,861, filed on Dec. 26, 2013.

(51) Int. Cl. A61K 39/00; C12N 5/0783

(52) U.S. Cl. CPC A61K 39/0011; C12N 5/0636; C12N 2506/45; A61K 2039/5158; C12N 2501/515; C12N 2510/00

(57) ABSTRACT

Provided is a method for immune cell therapy, which comprises generating T cell progenitors from pluripotent stem cells bearing rearranged T cell receptor genes and transferring the T cell progenitors to a patient in need of the treatment. The pluripotent stem cells may be iPS cells (T-iPS cells) bearing rearranged T cell receptor genes. By administering T cell progenitors instead of mature T cells, effective and safe immune cell therapy can be achieved.

### TECHNICAL FIELD

The present application relates to an immunotherapy method. In detail, the present application relates to an immunotherapy method using T cell progenitors induced from pluripotent stem cells having rearranged T cell receptor genes. The present application also relates to a method for inducing T cell progenitors from the pluripotent stem cells.

### BACKGROUND ART

A new treatment method wherein an antigen-specific cytotoxic T cell is propagated and thus propagated cells are transferred to a patient in need of the treatment. The main target for this proposed treatment is cancer. For example, a large number of induced pluripotent stem (iPS) cells are generated from a cancer antigen specific cytotoxic T cell and then, T cells that can be used for the treatment of the patient are generated from the iPS cells. (Vizcardo et al., Cell Stem Cell 12, 31-36, 2013, the contents of this document is herein incorporated by reference) Hereinafter, iPS cells induced from T cells are referred to as “T-iPS cells”. The advantageous points of this method are:

i) All of the T cells generated from the T-iPS cells are specific to the original antigen,

ii) T cells generated from the T-iPS cells are fresh and healthy, and

iii) Desired T cells can be generated to unlimited extent by multiplying the T-iPS cells.

T-iPS cells inherit the rearranged TCR configuration of the genes that are the same as TCR gene configuration of the original cancer antigen specific cytotoxic T cell. T cells induced from the T-iPS cells also inherit the specificity to the cancer antigen.

In the conventional adoptive immunotherapy technique in which in vitro propagated T cells are transferred to the patient, repeatedly sub-cultured T cells are used. The sub-cultured T cells could maintain only weak cytotoxic activities. By using T-iPS cells, a large number of fresh and healthy regenerated T cells can be provided.

### REFERENCES

### Patent Literature

[Patent Literature 1] Vizcardo et al., Cell Stem Cell 12, 31-36, 2013. This document is herein incorporated by reference.

### SUMMARY OF THE INVENTION

There is still a room for improvement in the above discussed immune cell therapy using T cells induced from T-iPS cells. The following issues remain unsolved.

i) There is a possibility that a secondary TCRα gene rearrangement occurs during the process for inducing T cells from T-iPS cells to give T cells with specificities are different from the original T cells. Said T cells could include dangerous autoreactive T cells.
ii) It has not yet been confirmed whether or not in vitro regenerated T cells are as healthy as the naive T cells generated in the thymus,
iii) Generating enough amount T cells for treating one patient will be fairly costly due to the troublesome tasks
iv) As suggested in above i), autoreactive T cells could be generated by unexpected secondary rearrangement of the TCRα chain. Even if the original TCR genes are maintained, the T cells generated from T-iPS cells could evoke dangerous reactions. The original T cell from which the T-iPS cells were induced had survived from the negative selection in the thymus or peripheral tolerance and therefore, is expected to have no auto-reactivity. However, there is a possibility that the original T-cell is an autoreactive T-cell and had survived accidentally. Further, when the patient or recipient is different from the donor of the original T cell from which the T-iPS cells were induced, considerably high risk that the regenerated T cells attack the normal tissue of the patient is expected.

Provided is a cell based therapy in which hematopoietic progenitor cells having an ability to develop into T cells, instead of mature T cells, are transferred to the patient. In this specification and claims, hematopoietic progenitor cells having an ability to develop into T cells are referred to as “T cell progenitors”. When T cell progenitors are transferred to a patient, the cells migrate into the thymus and differentiate into T cells there. Accordingly, even if the regenerated T cells are reactive against the patient's tissue, said reactive T cells will be removed in the thymus and therefore, this method is safe. In addition, the final stage of differentiation of T cell progenitors into mature T cells occurs in the patient's body and therefore, high quality regenerated T cells are produced from a relatively small number of T cell progenitors. Accordingly, this therapy can be conducted with relatively low cost.

In particular, this application provides followings:

[1] A method for generating T cell progenitors for immune cell therapy, which comprises the steps of differentiating pluripotent stem cells bearing rearranged T cell receptor genes into T cell progenitors in vitro.
[2] The method of [1], wherein the T cell progenitors are selected from the group consisting of CD34⁺CD5⁺CD4⁻CD8⁻ cells, CD34⁺CD38⁻CD45RA⁻CD10⁻ cells, CD45RA⁺CD10⁺CD7⁻CD5⁻ cells, CD45RA⁺CD10⁺CD7⁺CD5⁻ cells, CD45RA⁺CD10⁺CD7⁺CD5⁺ cells, CD3⁻CD4⁺CD8⁻ cells and CD4⁺CD8⁺ cells.
[3] The method of [1], wherein the pluripotent stem cells bearing rearranged T cell receptor genes are iPS cells induced from a human cytotoxic T cell.
[4] The method of [3], wherein the human cytotoxic T cell is induced from human peripheral mononuclear cells (PBMCs) by stimulating the PBMCs with an antigen.
[5] The method of [4], wherein the antigen is a cancer antigen and the immune cell therapy is for the treatment of a cancer patient.
[6] An immune cell therapy method, which comprises generating T cell progenitors from pluripotent stem cells bearing rearranged T cell receptor genes and transferring the T cell progenitors to a patient in need of the treatment.
[7] The immune cell therapy method of [6], wherein the T cell progenitors are selected from the group consisting of CD34⁺CD5⁺CD4⁻CD8⁻ cells, CD34⁺CD38⁻CD45RA⁻CD10⁻ cells, CD45RA⁺CD10⁺CD7⁻CD5⁻ cells, CD45RA⁺CD10⁺CD7⁺CD5⁻ cells, CD45RA⁺CD10⁺CD7⁺CD5⁺ cells, CD3⁻CD4⁺CD8⁻ cells and CD4⁺CD8⁺ cells.
[8] The immune cell therapy method of [6] or [7], wherein the pluripotent stem cells bearing rearranged T cell receptor genes are iPS cells induced from a human cytotoxic T cell.
[9] The immune cell therapy method of [8], wherein the human cytotoxic T cell is induced from human peripheral mononuclear cells (PBMCs) by stimulating the PBMCs with an antigen.
[10] The immune cell therapy method of [9], wherein the antigen is a cancer antigen and the patient to be treated is a patient suffered from cancer.

### BRIEF DESCRIPTION OF DRAWINGS

FIG. 1 is a schematic representation of the immune cell therapy against cancer, one embodiment of the present application.

FIG. 2 is FACS profile of T cell progenitors derived from iPS cells in Example A.

FIG. 3 is a schematic representation of Example B.

FIG. 4 is a schematic representation of Example C.

FIG. 5 shows antigen-specific killer activity of LMP2 specific CTLs that were used for inducing iPS cells in Example C.

FIG. 6 shows FACS profile of the cells obtained by co-culturing T cell progenitors bearing LMP2 antigen specific TCR genes with thymus tissue expressing human HLA-A2402⁺ in Example C.

FIG. 7 shows killer activity of CD8⁺ cells obtained in Example C by co-culturing T cell progenitors bearing LMP2 antigen specific TCR genes with thymus tissue expressing human HLA-A2402. The CD8⁺ cells were co-cultured with antigen presenting cells in the presence of LMP2 peptide.

### DETAILED DESCRIPTION

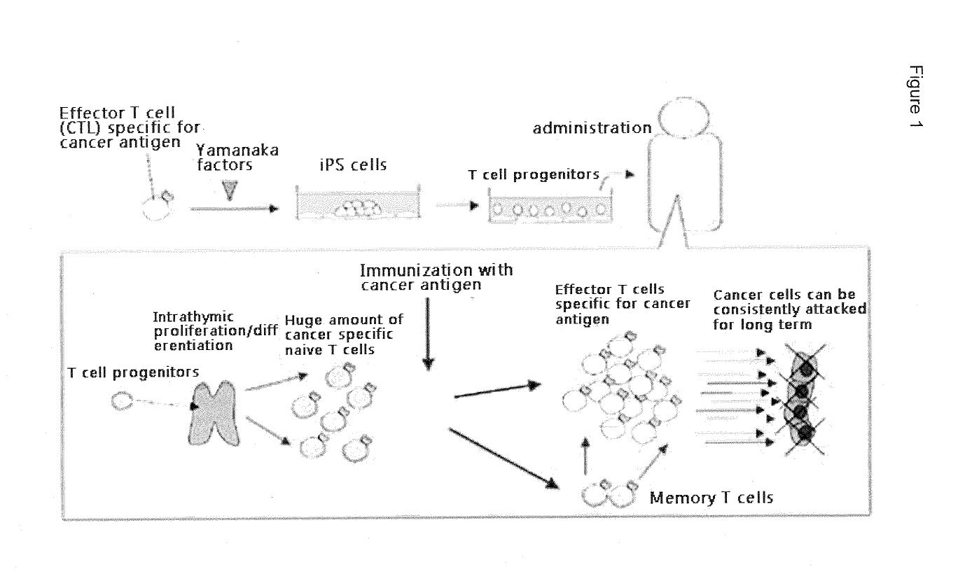

In one aspect, an immune cell therapy in which tumor antigen specific T cell progenitors are induced and transferred to a cancer patient is provided. One embodiment of the immune cell therapy is explained with referring to FIG. 1.

Firstly, iPS cells are induced from a tumor antigen specific cytotoxic T cell (CTL) according to a known procedure. T cell progenitors are induced from thus obtained iPS cells, herein after referred to as “T-iPS cells”, in vitro. The obtained T cell progenitors are transferred to the cancer patient.

The cells transferred to the patient migrate into the thymus and differentiate into mature T cells (naive T cells). The obtained mature T cells bear the rearranged T cell receptor genes. The naive T cells are activated to give tumor antigen specific CTL when the cells are stimulated with the tumor antigen that was used to induce the CTL cells as the source for the T-iPS cells. The activated antigen specific CTL cells specifically attack against the tumor.

In the specification and claims, “T cell progenitors” may cover cells at any stages of the T cell development, from the undifferentiated cells corresponding to hematopoietic stem cells to the cells at a stage just before the cells undergo positive selection/negative selection. T cell progenitors are explained in more detail.

Hematopoietic stem cells in human bone marrow are in general identified as CD34⁺CD38⁻CD45RA⁻CD10⁻ cells. In the specification and claims “+” means the cell expresses the gene and “−” does not. During the differentiation of the cells into T cells, the hematopoietic stem cells differentiate into CD45RA⁺CD10⁺ cells that have destiny of differentiating into B-T myeloid cells, then, sequentially express CD7 and CD5. At the stage where the cells express CD7, the cells are capable of differentiating into the T cells. The most undifferentiated T cell progenitors in the thymus are CD34⁺CD5⁺CD4−CD8− cells and said cells differentiate in the thymus to give CD3−CD4⁺CD8− and then, CD4⁺CD8⁺ cells. CD4⁺CD8⁺ cells express TCR and are subjected to the positive and negative selections, and then, become mature CD3⁺CD4⁺CD8⁻ cells or CD3⁺CD4⁻CD8⁺ cells. Accordingly, in the present specification and claims, the term “T cell progenitors” include from the stage of CD34⁺CD38⁻CD45RA⁻CD10⁻ cells to CD4⁺CD8⁺ cells. The development of T cells is explained in, for example, Blood 111:1318(2008) and Nature Immunology 11: 585(2010). The contents of the documents are herein incorporated by reference.

In one embodiment, pluripotent stem cells having the rearranged T cell receptor gene are differentiated into T cell progenitors. During the induction of hematopoietic cells from pluripotent stem cells, the way of development may not be the same as those found in the bone marrow. However, pluripotent stem cells may be developed through similar stages as the in vivo development of T cells. Accordingly, the “T cell progenitors” in the context of in vitro development of pluripotent stem cells my cover from the cells corresponding to the stages from hematopoietic stem cells to Cd4⁺CD8⁺ cells. More specifically, T cell progenitors may include, but not limited to, CD34⁺CD5⁺CD4⁻CD8⁻ cells, CD34⁺CD38⁻CD45RA⁻CD10⁻ cells, CD45RA⁺CD10⁺CD7⁻CD5⁻ cells, CD45RA⁺CD10⁺CD7⁺CD5⁻ cells, CD45RA⁺CD10⁺CD7⁺CD5⁺ cells, CD3⁻CD4⁺CD8⁻ cells and CD4⁺CD8⁺ cells. Preferably, CD45RA⁺CD10⁺CD7⁺CD5⁺ cells may be employed as T cell progenitors.

Pluripotent stem cells having antigen specific T cell receptor genes may be generated by known procedures. (Vizcardo et al., Cell Stem Cell 12, 31-56 2013, the contents of this document are herein incorporated by reference.) In particular, cytotoxic T cells specific for an antigen are prepared and the reprogramming factors are introduced into the cytotoxic T cells to give iPS cells.

Induced pluripotent stem (iPS) cells can be prepared by introducing specific reprogramming factors to somatic cells. iPS cells are somatic cell-derived artificial stem cells having properties almost equivalent to those of ES cells (K. Takahashi and S. Yamanaka (2006) Cell, 126:663-676; K. Takahashi et al. (2007), Cell, 131:861-872; J. Yu et al. (2007), Science, 318:1917-1920; Nakagawa, M. et al., Nat. Biotechnol. 26:101-106(2008); and WO 2007/069666). The reprogramming factors may be constituted by genes or gene products thereof, or non-coding RNAs, which are expressed specifically in ES cells; or genes or gene products thereof, non-coding RNAs or low molecular weight compounds, which play important roles in maintenance of the undifferentiated state of ES cells.

Examples of genes included in the reprogramming factors include Oct3/4, Sox2, Soxl, Sox3, Soxl5, Soxl7, Klf4, Klf2, c-Myc, N-Myc, L-Myc, Nanog, Lin28, Fbxl5, ERas, ECAT15-2, Tell, beta-catenin, Lin28b, Sall1, Sall4, Esrrb, Nr5a2, Tbx3 and Glis1, and these reprogramming factors may be used either individually or in combination. Examples of the combination of the reprogramming factors include those described in WO2007/069666; WO2008/118820; WO2009/007852; WO2009/032194; WO2009/058413; WO2009/057831; WO2009/075119; WO2009/079007; WO2009/091659; WO2009/101084; WO2009/101407; WO2009/102983; WO2009/114949; WO2009/117439; WO2009/126250; WO2009/126251; WO2009/126655; WO2009/157593; WO2010/009015; WO2010/033906; WO2010/033920; WO2010/042800; WO2010/050626; WO 2010/056831; WO2010/068955; WO2010/098419; WO2010/102267; WO 2010/111409; WO 2010/111422; WO2010/115050; WO2010/124290; WO2010/147395; WO2010/147612; Huangfu D, et al. (2008), Nat. Biotechnol., 26: 795-797; Shi Y, et al. (2008), Cell Stem Cell, 2: 525-528; Eminli S, et al. (2008), Stem Cells. 26:2467-2474; Huangfu D, et al. (2008), Nat Biotechnol. 26: 1269-1275; Shi Y, et al. (2008), Cell Stem Cell, 3, 568-574; Zhao Y, et al. (2008), Cell Stem Cell, 3:475-479; Marson A, (2008), Cell Stem Cell, 3, 132-135; Feng B, et al. (2009), Nat Cell Biol. 1 1:197-203; R. L. Judson et al. (2009), Nat. Biotech., 27:459-461; Lyssiotis C A, et al. (2009), Proc Natl Acad Sci USA. 106:8912-8917; Kim J B, et al. (2009), Nature. 461:649-643; Ichida J K, et al. (2009), Cell Stem Cell. 5:491-503; Heng J C, et al. (2010), Cell Stem Cell. 6: 167-74; Han J, et al. (2010), Nature. 463:1096-100; Mali P, et al. (2010), Stem Cells. 28:713-720, and Maekawa M, et al. (2011), Nature. 474:225-9. The contents of the documents cited in this paragraph are herein incorporated by reference.

The reprogramming factors may include factors for enhancing the establishment efficiency such as histone deacetylase (HDAC) inhibitors [e.g., low-molecular inhibitors such as valproic acid (VPA), trichostatin A, sodium butyrate, MC 1293, and M344, nucleic acid-based expression inhibitors such as siRNAs and shRNAs against HDAC (e.g., HDAC1 siRNA Smartpool® (Millipore), HuSH 29mer shRNA Constructs against HDAC1 (OriGene) and the like), and the like], MEK inhibitor (e.g., PD184352, PD98059, U0126, SL327 and PD0325901), Glycogen synthase kinase-3 inhibitor (e.g., Bio and CHIR99021), DNA methyl transferase inhibitors (e.g., 5-azacytidine), histone methyl transferase inhibitors [for example, low-molecular inhibitors such as BIX-01294, and nucleic acid-based expression inhibitors such as siRNAs and shRNAs against Suv39h1, Suv39h2, SetDB1 and G9a], L-channel calcium agonist (for example, Bayk8644), butyric acid, TGFβ inhibitor or ALK5 inhibitor (e.g., LY364947, SB431542, 616453 and A-83-01), p53 inhibitor (for example, siRNA and shRNA against p53), ARID3A inhibitor (e.g., siRNA and shRNA against ARID3A), miRNA such as miR-291-3p, miR-294, miR-295, mir-302 and the like, Wnt Signaling (for example, soluble Wnt3a), neuropeptide Y, prostaglandins (e.g., prostaglandin E2 and prostaglandin J2), hTERT, SV40LT, UTF1, IRX6, GLIS1, PITX2, DMRTB1 and the like. In the specification and claims, the term “reprogramming factors” may include not only the factors necessary for establishing iPS cells, but also the factors for enhancing the efficiency for establishing iPS cells.

In the cases where the reprogramming factors are proteins, each reprogramming factor may be introduced into somatic cells by means of, for example, lipofection, fusion with a cell-permeable peptide (e.g., HIV-derived TAT or polyarginine), or microinjection.

In the cases where the reprogramming factors are DNAs, each reprogramming factor may be introduced into somatic cells by a vector such as virus, plasmid and artificial chromosome vectors; or by means of lipofection, liposome or microinjection. Examples of the virus vectors include retrovirus vectors, lentivirus vectors (these are described in Cell, 126, pp. 663-676, 2006; Cell, 131, pp. 861-872, 2007; and Science, 318, pp. 1917-1920, 2007), adenovirus vectors (Science, 322, 945-949, 2008), adeno-associated virus vectors and Sendai virus vectors (WO 2010/008054). Examples of the artificial chromosome vector include human artificial chromosome (HAC), yeast artificial chromosome (YAC), and bacterial artificial chromosome (BAC and PAC). Examples of the plasmid which may be used include plasmids for mammalian cells (Science, 322:949-953, 2008). The vector may contain a regulatory sequence(s) such as a promoter, enhancer, ribosome binding sequence, terminator and/or polyadenylation site to enable expression of the reprogramming factors. If desired, the vector may also contain a selection marker such as a drug resistance gene (e.g., kanamycin-resistant gene, ampicillin-resistant gene or puromycin-resistant gene), thymidine kinase gene or diphtheria toxin gene; and a reporter such as the green-fluorescent protein (GFP), β-glucuronidase (GUS) or FLAG. Further, in order to remove the genes encoding the reprogramming factors, or the promoter(s) and the genes encoding the reprogramming factors linked thereto from the somatic cells after introduction and expression of the genes in the somatic cells, the vector may have LoxP sequences upstream and downstream of these sequences. The contents of the documents cited in this paragraph are herein incorporated by reference.

Further, in the cases where the reprogramming factors are RNAs, each reprogramming factor may be introduced into somatic cells by means of lipofection or microinjection. An RNA into which 5-methylcytidine and pseudouridine (TriLink Biotechnologies) were incorporated may be used in order to suppress degradation (Warren L, (2010) Cell Stem Cell. 7:618-630). The documents cited in this paragraph are herein incorporated by reference.

Examples of the medium for generating iPS cells include DMEM, DMEM/F12 and DME media supplemented with 10 to 15% FBS. Those media may further contain, for example, LIF, penicillin/streptomycin, puromycin, L-glutamine, non-essential amino acids, and/or β-mercaptoethanol as appropriate. The medium may be a commercially available medium, for example, medium for culturing mouse ES cells (TX-WES medium, Thromb-X), medium for culturing primate ES cells (medium for primate ES/iPS cells, ReproCELL) and serum-free medium (mTeSR, Stemcell Technology).

In order to generate iPS cells from somatic cells, for example, somatic cells in DMEM or DMEM/F12 medium supplemented with 10% FBS at 37° C. and 5% CO2 are contacted with reprogramming factors, and cultured for about 4 to 7 days. Then, the cells are seeded on the feeder cells such as mitomycin C-treated STO cells or SNL cells. About 10 days after the contact with reprogramming factors, the cells are transferred to a medium for primate ES cells supplemented with bFGF and cultured further. ES cell-like cell colonies will be observed at around 30 to around 45 days after the contact or later.

Alternatively, the cells may be contacted with the reprogramming factors and cultured at 37° C. and 5% CO₂ on feeder cells (e.g., mitomycin C-treated STO cells or SNL cells) in DMEM medium supplemented with 10% FBS (this medium may further contain LIF, penicillin/streptomycin, puromycin, L-glutamine, non-essential amino acids, β-mercaptoethanol and the like, as appropriate) for about 25 to about 30 days or longer, thereby allowing ES-like colonies to appear. Preferred examples of the culture method include a method wherein the somatic cells themselves to be reprogrammed are used instead of the feeder cells (Takahashi K, et al. (2009), PLoS One. 4:e8067 or WO2010/137746), and a method wherein an extracellular matrix (e.g., Laminin-5 (WO2009/123349), Laminin-10 (US2008/0213885) or its fragment (WO2011/043405) or Matrigel (BD)) is used instead. The documents cited in this paragraph are herein incorporated by reference.

Other examples include a method wherein the iPS cells are established using a serum-free medium (Sun N, et al. (2009), Proc Natl Acad Sci USA. 106: 15720-15725). Further, in order to enhance the establishment efficiency, iPS cells may be established under low oxygen conditions (at an oxygen concentration of 0.1% to 15%) (Yoshida Y, et al. (2009), Cell Stem Cell. 5:237-241 or WO2010/013845). The contents of the documents cited in this paragraph are herein incorporated by reference.

During the culture, the medium is replaced with the fresh medium once every day from Day 2 of the culture. The number of somatic cells used for nuclear reprogramming is not restricted, and usually within the range of about 5×10³ to about 5×10⁶ cells per 100 cm² area on the culture plate.

iPS cells may be selected based on the shape of each formed colony. In the cases where a drug resistance gene is introduced as a marker gene such that the drug resistance gene is expressed in conjunction with a gene that is expressed when a somatic cell was reprogrammed (e.g., Oct3/4 or Nanog), the established iPS cells can be selected by culturing the cells in a medium containing the corresponding drug (selection medium). Further, iPS cells can be selected by observation under a fluorescence microscope in cases where a gene of a fluorescent protein is introduced as a marker gene. iPS cells can also be selected by adding a luminescent substrate in the cases where a gene of a luminescent enzyme is introduced as a marker gene.

Cytotoxic T cells specific for an antigen may be generated by a known method. For example, cytotoxic T cells specific for a cancer antigen may be generated by stimulating lymphocytes derived from a human in a conventional manner with the cancer antigen specific for the cancer to be treated. Cancer antigens for various cancers have been identified. Cytotoxic T cells may be induced by using a cancer antigen or an epitope peptide of the antigen. Alternatively, the lymphocytes may be stimulated with the cancer cells to be treated. Further, the cytotoxic T cells specific for a cancer antigen that is specific for the cancer to be treated may be derived from a donor who is suffered from the cancer.

Thus obtained cytotoxic T cells may be induced into iPS cells by introducing the reprogramming factors, for example, the Yamanaka factors, into the cytotoxic T cells. In order to enhance the establishment efficiency, SV40 may be added to the Yamanaka factors. Thus induced iPS cells bear the rearranged T cell receptor genes derived from the original cytotoxic T cell specific for the cancer antigen. In the specification and claims, the iPS cells bearing the rearranged T cell receptor genes are referred to as “T-iPS cells”.

T cells regenerated from T-iPS cells can be used in the treatment of a subject other than the donor of the cytotoxic T cell from which the T-iPS cells were generated. For example, banks of T-iPS cell lines generated from cytotoxic T cells obtained from donors having specific HLA haplotypes may previously be established. The donors may be patients suffered from a specific disease or healthy volunteers.

A project establishing iPS cell bank having high versatility is in progress. This project uses somatic cells obtained from healthy HLA homozygous donors, whose HLA haplotypes are frequently found in Japanese population, for generating the iPS cells (CURANOSKI, Nature vol. 488, 139(2012)), the contents of the document is herein incorporated by reference. A T-iPS cell bank having high versatility can also be established by using leucocytes derived from the same type donors as above. That is, cytotoxic T cells specific for the cancer antigen which is specific for the cancer to be treated may be induced from leucocytes, and T-iPS cells may be induced from said cytotoxic T cells, and a bank of thus induced T-iPS cells may be established.

In the T-iPC cell bank, all cell lines should be registered with information regarding donor's HLA and the antigen.

A suitable T-iPS cell line can be selected based on HLA of the patient and the type of the cancer to be treated. The selected T-iPS cell line is differentiated into T cell progenitors and the T cell progenitors may be administered to the patient. Alternatively, T cell progenitors, but not T-iPS cells, may be frozen and used to establish T-progenitor cell Bank so that quick treatment can be provided to patients.

In the immune cell therapy provided by the present application, the induced T cell progenitors may be suspended in a suitable vehicle such as saline or PBS and administered to the patient provided that the degree of HLA matching between the patient and the donor is higher than a certain fixed value. In this contest, “the degree of HLA matching between the patient and the donor is higher than a certain fixed value” covers the situations where HLAs are completely identical between the patient and the donor, and also where HLAs match to the extent that taking of the transplantation is expected even one or two loci mismatches are present. For example, when the donor has homozygous HLA and either one of the patient's HLA alleles matches to the donor's HLA, then the T-iPS cells derived from the donor can be used for the patient.

In the specification and claims, “multipotent stem cells having rearranged T cell receptor genes” are not limited to T-iPS cells obtained by the above-explained procedure. Said cells may be any types of pluripotent stem cells that can differentiate into various tissues or organs and proliferate indefinitely in culture, and bear the rearranged T cell receptor genes. For example, cells obtained by introducing the rearranged T cell receptor genes into ES cells or iPS cells may be used. The introduction of TCR genes into pluripotent stem cells may be conducted by a known method, for example, by a method taught in Mrgan R. A. et al, Science 314:126 2006 (the contents of this document is herein incorporated by reference). Various T cell receptor genes specific for various antigens have been known to the art. For example, TCR genes specific for EB virus related antigen are disclosed in Jurgens et al, Journal of Clinical Investigation, 26:22, 2006, the contents of this document are herein incorporated by reference. TCR genes specific for WT1 related antigens are disclosed in, for example, Anticancer Research 32(12); 5201-5209, 2012, the contents of this document is herein incorporated by reference.

The pluripotent stem cells bearing rearranged TCR genes are differentiated into T cell progenitors. The differentiation may be conducted according to the procedures disclosed in, for example, Anticancer Research 32(12); 5201-5209, 2012, the contents of this document are herein incorporated by reference. Specifically, the pluripotent stem cells and OP9 stromal cells, such as mouse OP9 stromal cells, are co-cultured to give hematopoietic progenitor cells, and the hematopoietic progenitor cells are then co-cultured with OP9 stromal cells and DLL1 cells. The co-culture with OP9/DLL1 cells may be conducted in a medium supplemented with IL-7, FLT-3L and SCF (Stem Cell Factor).

Although the quoted document explains the procedure for differentiating ES cells into mature T cells, T cell progenitors obtained prior to the mature T cells can be used herein. T cell progenitors may be identified by the cell surface markers. The cell surface markers may be determined by a conventional method. Examples of T cell progenitors may include:

CD34⁺CD5⁺CD4⁻CD8 cells, CD34⁺CD38⁻CD45RA⁻CD10⁻ cells, CD45RA⁺CD10⁺CD7⁻CD5⁻ cells, CD45RA⁺CD10⁺CD7⁺CD5⁻ cells, CD45RA⁺CD10⁺CD7⁺CD5⁺ cells, CD3⁻CD4⁺CD8⁻ cells and CD4⁺CD8⁺ cells.

Thus obtained T cell progenitors are administered to the patient. T cell progenitors may be suspended in a suitable vehicle such as saline or PBS before administering to the patient. The T cell progenitors may be administered intravenously or intrathymically.

The number of T cell progenitors to be administered is not specifically limited and may be determined based on, for example, age, sex, height and body weight of the patient or disease and conditions to be treated. For example, 10⁶-10⁸ T cell progenitors may be administered intravenously, and 10⁵-10⁷ T cell progenitors may be administered intrathymically to an adult male cancer patient who weighs about 70 kilograms. Optimal amount of the cells to be administered may be determined by clinical studies.

The T cell progenitors administered to the patient migrate into thymus and develop there into mature T cells or naive T cells. Thus generated mature T cells bear the rearranged TCR genes. The naive T cells may be stimulated by the cancer antigen used for developing the cytotoxic T cells used as the source of the T-iPS cells. The stimulated naive T cells are activated to give antigen specific cytotoxic T cells and attack specifically against the cancer. In order to stimulate the naive T cells, for example, the cancer antigen peptide may be administered to the patient.

According to the method provided herein, the followings can be achieved:

i) Safe procedure is provided

When T cell progenitors are administered to a patient, T cells are generated in the thymus. Even if auto-reactive T cells are generated there, they will be removed by negative selection.

ii) Re-generated T cells with improved quality are provided

The naive T cells generated in the thymus are expected healthy T cells that function properly.

iii) Treatment requires administration of a relatively small number of the T cell progenitors

It has been known that one T-progenitor cell that migrates into the thymus will generate at least 10⁶ immature T cells. The immature T cells are subjected to the positive and negative selections in the thymus and then, not more than about 5% of the immature T cells migrate into the peripheral as naive T cells. Among them, only less than 1/10,000 of T cells are reactive to a specific epitope. In contrast, when T cell progenitors generated from T-iPS cells are administered, almost all T cells generated in the thymus may survive the positive and negative selections in the thymus. The T cell progenitors generated from the T-iPS cells express TCR genes once survived from the selections in the thymus. In addition, thus generated T cells bear the antigen specificity of the original killer T cell. That is to say, one T-progenitor cell can generate 10⁶ antigen specific T cells. Hence, the number of the cells to be administered to the patient may be small and the cost can be reduced.

### EXAMPLES

Embodiments of this application will be explained in more detail based on the examples shown below. The examples are just for illustrate and do not restrict or limit the scope of the invention disclosed herein.

### Example A

Induction of T Cell Progenitors from iPS Cells

### Materials

OP9 cells and OP9/DLL1 cells: obtained from Riken BioResource Center (Tsukuba, Ibaraki pref. Japan)

Human iPS cells: established from umbilical cord blood hematopoietic progenitor cells in Riken Research center for allergy and immunology (Yokohama, Kanagawa pref. Japan). The human iPS cells used herein could also be established by the method described in Vizcardo et al., Cell Stem Cell, 12:31-36, 2013.

Media used are as follows:

TABLE 1
Medium A for maintenance of OP9 stromal cells

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 6.25 | mL | 1% |
| solution* | | | |
| Total | 631.25 | mL | |

*Mixture of Penicillin (10,000 U/ml) and Streptomycin (10,000 μg/ml). The final concentrations were 100 U/ml and 100 μg/ml, respectively.

TABLE 2
Medium B for inducing differentiation of T cells (T cell medium)

| | | |
|---|---|---|
| contents | amount added | final conc. |

| | | | |
|---|---|---|---|
| αMEM medium | 500 | mL | |
| FCS | 125 | mL | 20% |
| penicillin-streptomycin | 5 | mL | 1% |
| solution* | | | |
| hrIL-7 (stock: 10 μg/mL) | 315 | μL | 5 ng/mL |
| hrFlT-3L (stock: 10 μg/mL) | 315 | μL | 5 ng/mL |
| hrSCF (stock: 10 μg/mL) | 630 | μL | 10 ng/mL |
| Total | 631.26 | mL | |

*Mixture of Penicillin (10,000 U/ml) and Streptomycin (10,000 μg/ml). The final concentrations were 100 U/ml and 100 μg/ml, respectively.

0) Preparation of OP9 Cells

Six milliliter (6 mL) of 0.1% gelatin solution was added to a 100 mm dish (Falcon) and incubated for 30 minutes at 37° C. The gelatin solution was then removed and 10 ml of medium A was added to the dish. OP9 stromal cells were obtained from a confluent culture and seeded in the dish. Four days after, medium A 10 mL was added to the dish (final amount was 20 mL).

1) Induction of Hematopoietic Progenitor Cells from iPS Cells

### Day 0: Co-Culture of iPS Cells and OP9 Stromal Cells

The medium in the OP9 stromal cell culture was replaced with fresh medium A. iPS cells were cultured in a 100 mm dish (Falcon) to form a confluent culture, the, the culture medium was replaced with fresh medium A 10 mL. The aggregated human iPS cells adhered to the bottom of the cell culture dish were separated by means of EZ-passage roller. Thus obtained iPS cell aggregates were dispersed by pipetting and the cells were suspended in medium A. The number of iPS cell-aggregates was visually counted and approximately 600 iPS cell-aggregates (about 1×10⁶ cells) were inoculated on the OP9 stromal cells.

Three dishes were prepared for one clone of human iPS cells. When passaging the iPS cells, the cells in the three dishes were combined to one dish and then divided into three fresh dishes to reduce difference among the dishes.

Day 1: (after One Day Had Passed, Replace the Medium)

The cell culture medium was replaced with fresh medium A 20 mL.

Day 5: (after 5 Days Had Passed, Replace a Half of the Medium)

A half of the cell culture medium was replaced with fresh medium A 10 mL.

Day 9: (after 9 Days Had Passed, Replace a Half of the Medium)

A half of the cell culture medium was replaced with fresh medium A 10 mL.

2) Induction of T Cells from Hematopoietic Progenitor Cells

Day 13: (after 13 Days Had Passed)

Up to this point, iPS cells had differentiated into mesodermal cells. Thus induced mesodermal cells were transferred from OP9 cell layer to OP9/DLL1 cell layer according to the procedures as follows:

Cell culture medium was sucked to remove and the surface of the cultured cells were washed with HBSS(⁺M⁺Ca) to washout the cell culture medium. Collagenase 250U in IV/HBSS (+Mg+Ca) solution 10 mL was added to the dish and incubated for 45 minutes at 37° C.

The collagenase solution was removed by sucking it and the cells were washed with PBS(−) 10 mL. Then, 0.05% trypsin/EDTA solution was added to the dish and the dish was incubated for 20 minutes at 37° C. After the incubation, the sheet like cell aggregates peeled from the bottom of the dish and the cell aggregates were mechanically fragmented to smaller sizes by means of pipetting. Thus treated cells were added with fresh medium A 20 mL and cultured for more 45 minutes at 37° C.

The culture medium containing the floating cells was passed through a 100 μm mesh and the cells were collected. The cells were then centrifuged at 1200 rpm for 7 minutes at 4° C. The obtained pellet was suspended in medium B 10 mL. One-tenth of the suspension was separated and used for the FACS analysis. The remaining cell suspension was seeded to new dishes containing OP9/DLL1 cells. Cell suspensions obtained from several dishes were pooled and the pooled cells were seeded to the same number of new dishes.

In order to confirm whether or not the obtained cells contain hematopoietic progenitor cells, the cells were subjected to flow cytometry analysis for CD34 and CD43. A considerable number of the cells were found within the CD34low/CD43⁺ population. Thus, it was confirmed that iPS cells were differentiated into hematopoietic stem cells.

Then, the obtained cells were seeded to new dishes containing OP9/DLL1 cells. In this step, the cells were not sorted for CD34lowCD43⁺ population. Sorting for CD34lowCD43⁺ population could lower the differentiation efficiency due to the decrease of the total cell number and damages during the sorting step.

During the culturing period, FACS analysis was conducted several times to confirm the differentiation stages. A considerable number of dead cells were observed over the culturing period. Before the FACS analysis, dead cells were eliminated by using, for example, Propidium Iodide (PI) or 7-AAD.

Day 16: (after 16 Days Had Passed, Subculture of the Cells)

The cells weakly adhered to the OP9 cells were gently dissociated by pipetting several times. The cells were passed through a 100 μm mesh and collected in a 50 mL conical tube. The tube was centrifuged at 1200 rpm for 7 minutes at 4° C. The pellet was dispersed in medium B 10 mL. Thus prepared cells were inoculated to a new dish containing OP9/DLL1 cells.

Day 23: (after 23 Days Had Passed, Subculture of the Cells) Blood Cell Colonies Began to Appear.

The cells weakly adhered to the OP9/DLL1 cells were gently dissociated by pipetting several times. The cells were passed through a 100 μm mesh and collected in a 50 mL conical tube. The tube was centrifuged at 1200 rpm for 7 minutes at 4° C. The pellet was dispersed in medium B 10 mL.

Day 30: (after 30 Days Had Passed, Subculture of the Cells) CD7⁺CD5⁺ Cells Began to Appear.

The OP9/DLL1 cells aggregated and adhered to the bottom of the dish, and were gray. The T cell progenitors weakly adhered to and propagated on the OP9 cells, and therefore, were bright color compared to the OP9 cells. An aggregated colony of T cell progenitors was not observed. Instead, aggregates of several bright-colored particles were observed.

The cells weakly adhered to the OP9/DLL1 cells were gently dissociated by pipetting several times. The cells were passed through a 100 μm mesh and collected in a 50 mL conical tube. The tube was centrifuged at 1200 rpm for 7 minutes at 4° C. The pellet was dispersed in medium B 10 mL. One-tenth of the suspension was separated and used for the FACS analysis. The remaining cell suspension was inoculated to new dishes containing OP9/DLL1 cells.

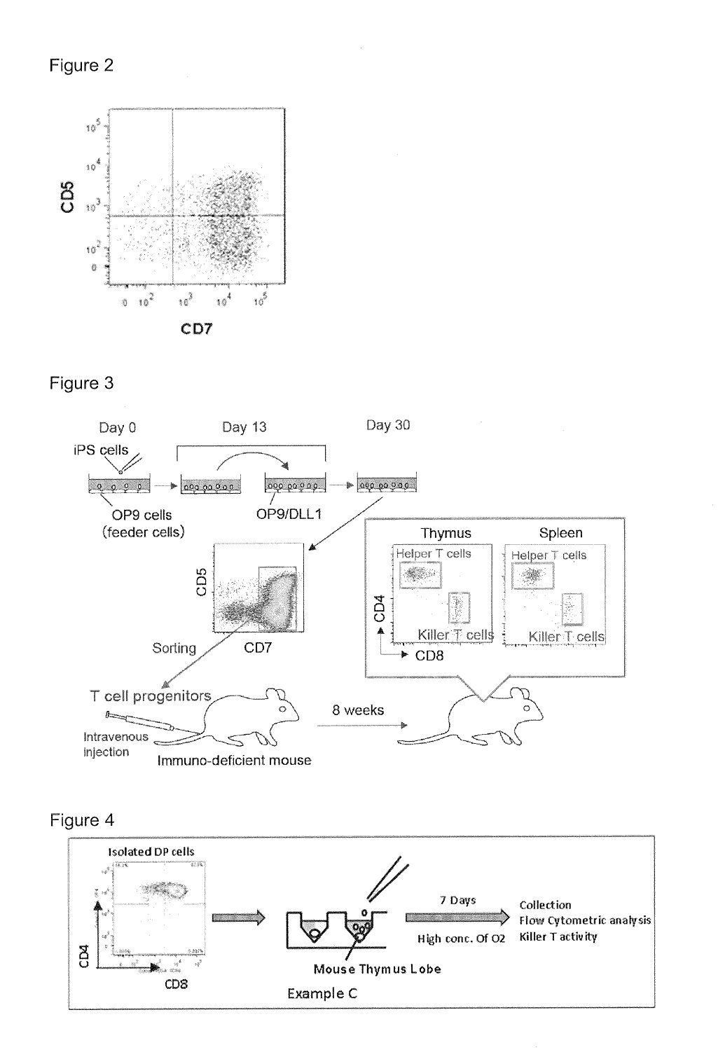

In order to determine whether or not T cell progenitors were induced, FACS analysis with anti-CD5 antibody and anti-CD7 antibody was conducted. CD7⁺ cells or T cell progenitors were observed and a part of the cells were differentiated to the CD7⁺CD5⁺ stage. Results of FACS analysis are shown in FIG. 2.

### Example B

Transplantation of T cell progenitors differentiated from iPS cells into immune deficient mice. T cell progenitors differentiated from iPS cells in Example A were transplanted into immune deficient NOG mice to show human T cells were generated. The human iPS cells were induced from CD34⁺ cells derived from human umbilical cord blood. The immune deficient NOG mice were purchased from Central Institute for Experimental Animals, Kawasaki city, Kanagawa, Japan.

1) Transplantation of T Cell Progenitors into Immune Deficient Mice

CD7⁺ T cell progenitors were isolated from the cell culture by using a cell sorter and 10⁵ cells were intravenously injected to the recipient mice.

2) Detection of Mature T Cells in the Recipient Mice.

The mice were sacrificed at 8 weeks after the transplantation and analyzed. Mature T cells, i.e. CD4⁺ T cells and CD8⁺ T cells were observed in the thymus and spleen of the mice.

3) Negative Selection Occurred in the Recipient Mice

No GVH reaction was observed in the recipient mouse. In general, Mice who receive transplantation of human peripheral blood T lymphocytes will die. In this example, however, no symptom was observed in the recipient mice. This result means that T cells reactive against a molecule in the body of recipient mouse were eliminated by negative selection. The protocol and results of this example are summarized in FIG. 3.

As was confirmed by preliminary tests, the administered T cell progenitors were differentiated into mature T cells in the thymus. During the differentiation, auto-reactive T cells were eliminated by negative selection.

### Example C

Organ culture of the T cell progenitors generated from T-iPS cells in mouse thymus lobe

In Examples A and B, T cell progenitors were generated from iPS cells bearing no rearranged TCR. In those examples, the T cell progenitors were injected to a mouse and the cells were differentiated into mature T cells in the mouse thymus. Thus generated polyclonal T cells did not attack against the recipient mouse. It was concluded that “negative selection” occurred in the thymus.

In example C, T-iPS cells were generated from antigen specific T cells and then, T cell progenitors were generated from the T-iPS cells. Thus obtained T cell progenitors were introduced in thymus lobe derived from human HLA transgenic mice and conducted organ culture. The protocol of example C is summarized in FIG. 4.

1) Preparation of LMP2-T-iPS Cells

T-iPS cells (LMP2-T-iPS cells) were induced from killer T cells that express HLA-A2402-restricted and LMP2 antigen specific TCR.

EB virus is a virus that causes infectious mononucleosis in the acute phase of infection and also causes cancer such as Burkitt's lymphoma. In this example, the donor of the T cells was a healthy person who had previously been infected by EB virus. Once infected, EB virus remains in the lymphatic system of the patient for life and therefore, the donor carried EB virus. That is, the donor did not have any symptom but had chronically been infected with the virus.

### a) Propagation of LMP2 Antigen Specific Cytotoxic T Lymphocytes

i) The following media were used.
Medium for dendritic cells: CellGro (CellGenix)
Medium for T cells:

TABLE 3

| | Amount | Final concentration |
|---|---|---|
| RPMI | 45 ml | |
| human AB serum | 5 ml | 10% |
| Total | 50 ml | |

ii) Sequence of LMP2 antigen peptide

### TYGPVFMSL

LMP2 tetramer was purchased from MBL.
iii) Antigen presenting cells

Lymphoblastoid cell line (LCL) having HLA-A2402 established from a healthy volunteer in Prof. Kadowaki's laboratory, the Department of Hematology and Oncology, Graduate School of Medicine, Kyoto University, Kyoto, Japan was used as antigen presenting cells.

A. Generation of Human Monocyte Dendritic Cells (MoDC) from Human Peripheral Blood
1. Peripheral blood was obtained from a healthy volunteer having HLA-A2402 and previously infected by EB virus. Monocytes were isolated from the blood by using CD14 microbeads. The cells were washed and added with the medium for dendritic cell culture to give 5×10⁵ cells/mL suspension.
2. Cytokines were added to the cell suspension to give final concentrations of GM-CSF 800 U/mL (or 50 ng/mL), IL-4 200 U/mL (or 40 ng/mL). Five milliliter (5 mL) of the cell suspension was seeded to each well of a 6-well plate. The plate was incubated at 37° C. with 5% CO₂.
3. The plate was incubated for 3 days and on day 3, 2.5 mL of the culture supernatant was gently removed.
4. Fresh medium for dendritic cell was added with GM-CSF and IL-4 to give final concentrations of 800 U/mL and 200 U/mL respectively.
5. Thus prepared fresh medium for dendritic cells 3 mL was added to each well.
6. On day 6, immature monocyte-derived dendritic cells (MoDCs) were collected from the plate and added in a small amount of fresh medium for dendritic cells to give 5×10⁵ cells/mL suspension.
7. GM-CSF (final concentration: 800 U/mL), IL-4 (final concentration: 200 U/mL), TNF-alpha (final concentration: 10 ng/mL), and PGE2 (final concentration: 1 μg/mL) were added to the cell suspension. About 5×10⁵ cells/mL/well of the cell suspension was seeded to each well of a 24-well plate.
8. The plate was incubated at 37° C. with 5% CO2 for 24 hours.
9. The peptide was added to each well in last 2 hours of 24 hours of the incubation period. The final concentration of the peptide was 10 μM.
10. Dendritic cells (DC) were collected from the plate and washed twice with the medium for T cells. The number of the DC cells was counted and the medium for T cells was added to give a 2×10⁵ cells/mL suspension.
B. Isolation of T Cells from Human Peripheral Blood and Co-Culture of the T Cells and Dendritic Cells.
1. T cells were isolated from peripheral blood of the same healthy volunteer as in above step A by means of the MACS technique using CD3 microbeads. The cells were washed and added with the medium for T cells to give 2×10⁶ cells/mL suspension. A small part of the T cell suspension was separated for the flow cytometer analysis.
2. 0.5 mL/well of DC cell suspension (2×10⁵ cells/mL) and 0.5 mL/well of T cell suspension (2×10⁶ cells/mL) were added to each well of a 24 well plate. (DC cells: T cells=1×10⁵: 1×10⁶=1:10).
3. On day 3, IL-7 (final concentration: 5 ng/mL) and IL-15 (final concentration: 10 ng/mL) were added to each well.
4. On day 14, the cells were collected from the culture.

### C. Addition of the Peptide to LCL

1. LCLs were collected from the culture and irradiated at a dose of 35Gy.
2. The irradiated cells were suspended in the T cell medium to give 5×10⁵ cells/mL suspension.
3. 100 nM of the peptide was added to the suspension and incubated for 2 hours.
4. The LCL were collected and washed with the T cell medium and then, dispersed in the T cell medium to give 2×10⁵ cells/mL suspension.

### D. Co-Culture of LCL and T Cells Stimulated by the Dendritic Cells.

1. The T cells stimulated with the dendritic cells were dispersed in the T cell medium to give a 2×10⁶ cells/mL suspension.
2. 0.5 mL/well of LCL suspension (2×10⁵ cells/mL) incubated in the presence of the peptide and 0.5 mL/well of T cell suspension (2×10⁶ cells/mL) were added to each well of a 24-well plate (LCL: T cells=1×10⁵: 1×10⁶=1:10). Simultaneously, the peptide was added to the well to give the final concentration of 100 nM.
3. On day 3, IL-7 (final concentration: 5 ng/mL) and IL-15 (final concentration: 1 ng/mL) were added to the well. The plate was incubated for 2 weeks and the medium was changed with the fresh T cell medium supplemented with the cytokines for every one week. (1st course of stimulation with peptide-pulsed LCL)
4. LCLs were again incubated in the medium supplemented with 100 nM of the peptide for 2 hours and then, added with the cells obtained in step 3.
5. On day 3, IL-7 (final concentration: 5 ng/mL) and IL-15 (final concentration: 1 ng/mL) were added to the well. The plate was incubated for 2 weeks and the medium was changed with the fresh T cell medium supplemented with the cytokines for every one week. (2st course of stimulation with peptide-pulsed LCL)
6. Thus obtained cells were analyzed by flow cytometer and confirmed that more than 80% of CD8 positive T cells were CD8 positive and LMP-2 tetramer positive cells.

### E. Antigen Specific Killer Activity of the LMP2 Specific CTLs

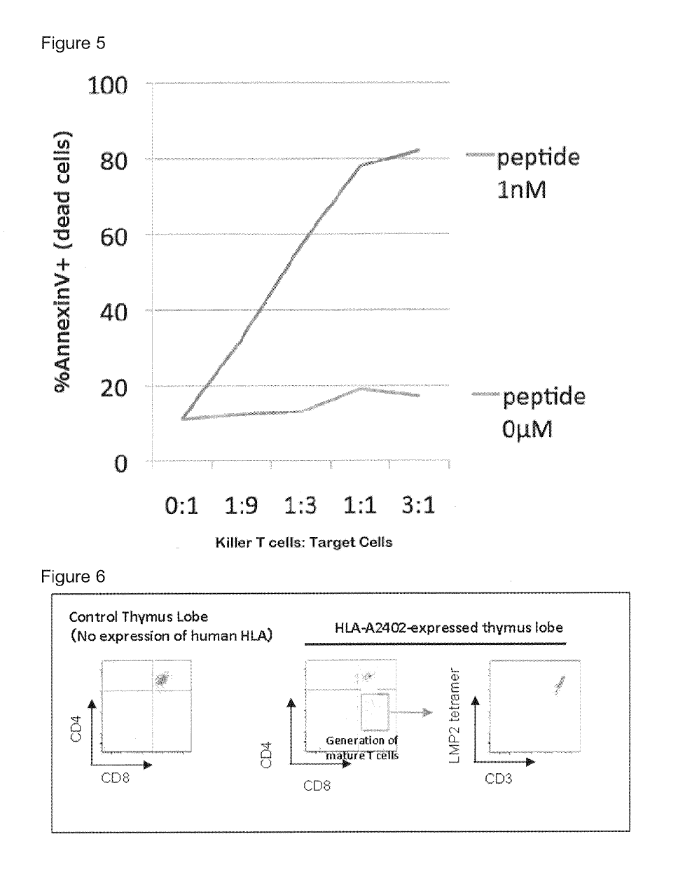

1. CFSE-labelled OUN-1 leukemia cells were used as target cells. The labelled cells were dispersed in the T cell medium and incubated in the presence of 1 nM of the LMP2 peptide.
2. LMP2 specific cytotoxic T cells (CD8 positive and LMP-2 tetramer positive cells) expanded under the peptide stimulation and the CFSE-labelled OUN-1 leukemia cells were added to each well of a 96-well round bottom plate at different effector/target cell ratios of 0:1, 1:9, 1:3, 1:1 and 3:1. The cells were incubated in the presence or absence of the peptide. The ratio of Annexin V positive cells to PI (Propidium Iodide) positive cells among the CFSE positive sorted cells were determined to confirm death rate of the target cells. Results are shown in FIG. 5.
3. Thus prepared LMP2 specific killer T cells were confirmed to have the antigen specific killer activity against the target cells.

### b) Establish of the LMP2-T-iPS Cells

### A. Activation of LMP2 Specific CTLs.

1. CD8 positive cells were enriched from the above obtained LMP2 specific CTLs using MACS beads.
2. The enriched cell population was dispersed in the T cell medium and added with IL-7 (final concentration: 5 ng/mL) and IL-15 (final concentration: 10 ng/mL). Dynabeads Human T-Activator CD3/CD28 was added to give a bead-to-cell ratio of 1:1, and the mixture was incubated for 2 days to activate the CD8 positive cells.

### B. Introduction of the Yamanaka Four Factors and SV40 by Means of Sendai Virus Vector.

1. The activated LMP2 specific CTLs were dispersed in the T cell medium, Sendai virus containing four Yamanaka factors and SV40 was added to the medium and the cell suspension was cultured for 2 days.
2. The obtained cells were washed with the T cell medium and added with the T cell medium supplemented with IL-7 (final concentration: 5 ng/mL) and IL-15 (final concentration: ing/mL). The cells further cultured for 2 days.
3. After that, all cells were collected and dispersed in the T cell medium supplemented with IL-7 (final concentration: 5 ng/mL) and IL-15 (final concentration: 1 ng/mL). The cell suspension was seeded on the feeder cells.
4. On day 2, a half of the T cell medium was replaced with fresh iPS cell medium. After day 2, a half of the medium was replaced with fresh iPS cell medium every day and the cells were continued to be cultured.
C. Picking Up iPS Cell Colonies from the Culture
1. Three weeks after the introduction of the Yamanaka factors, colonies of iPS cells were visually observed.
2. Colonies were mechanically picked up with a 200 μl pipette tip.
3. Several clones were established individually and one of them was used as LMP2-T-iPS cells in the example below.
2) Induction of T cell progenitors from the LMP2-T-iPS cells. The LMP2-T-iPS cells were treated according to the protocol of Example A and CD4CD8 double positive cells (DP cells) were obtained on day 35-40.

### 3) Mouse Thymus Organ Culture of T Cell Progenitors

Mouse thymus organ culture was conducted according to the procedures disclosed in Kawamoto et al, International Immunology, 9:1011 (1997), the content of this document is herein incorporated by reference.

HLA-A2402 transgenic C57BL6 mouse (CB6F1-Tg(HLA-A*2402/H2-Kb)A24.01) was purchased from Taconic Biosciences, Inc. Normal C57BL6 mouse was used as control mice.

In order to obtain an immune deficient recipient mouse, the HLA-A2402 transgenic C57BL6 mouse or normal C57BL6 mouse was crossbred with Rag2KO mouse. The fetuses were removed from the pregnant mouse at a gestational age of 15 days and the thymus lobes of the fetuses were isolated. The fetal thymus lobes were cultured for 6 days on filters floating on RPMI1640 medium supplemented with deoxyguanosine (dGuo). By this treatment, the endogenous thymocytes were killed and cells within the thymic environment remained.

Each one of thus obtained dGuo treated fetal thymus lobes was placed in each well of a 96-well V-bottom plate, and added with 0.2 ml RPMI1640 medium. DP cells purified with MACS beads were added to the wells. The plate was placed in a plastic bag and the air in the bag was exchanged with a gas mixture of 5% CO₂, 25% N₂ and 70% O₂. The DP cells and dGuo treated thymus lobe were co-cultured under a high oxygen condition by placing the plastic bag in a 37° C. incubator.

On day 7 of co-culture, the cultured material was took out and squeezed between two slide glasses. The crushed cultured material was dispersed in a medium and T cells obtained as floating cells in the medium were collected. Thus obtained T cells were analyzed.

Flow Cytometric Analysis

Thus obtained T cells were subjected to flow cytometric analysis. T cells obtained by co-culturing with the thymus lobe derived from the control mouse, the added cells were at the stage of DP. On the other hand, T cells obtained by co-culturing with the thymus lobe derived from the human HLA-2402 expressing mouse, mature T cells were generated. Almost all of the T cells generated were LMP2 tetramer positive, i.e. expressed the original TCR. See FIG. 6.

Measurement of the Killer Activity

Lymphoblastoid cell line (LCL, B-lymphoblastic cell line) established from a healthy volunteer having HLA-A2402, gifted from prof. Kadowaki's laboratory, the Department of Hematology and Oncology, Graduate School of Medicine, Kyoto University, Kyoto, Japan was used as antigen presenting cells.

THP1 cells (Human acute monocytic leukemia cell line) gifted from prof. Kadowaki's laboratory, the Department of Hematology and Oncology, Graduate School of Medicine, Kyoto University, Kyoto, Japan were used as target cells. LMP2 antigen peptide: TYGPVFMSL(419-427)

Medium: αMEM medium (life technologies, cat #11900-073) supplemented with 20% bovine fetal serum.

The generated CD8 positive cells were isolated and the cells were cultured in the presence of the antigen presenting cells and LMP2 peptide for 13 days.

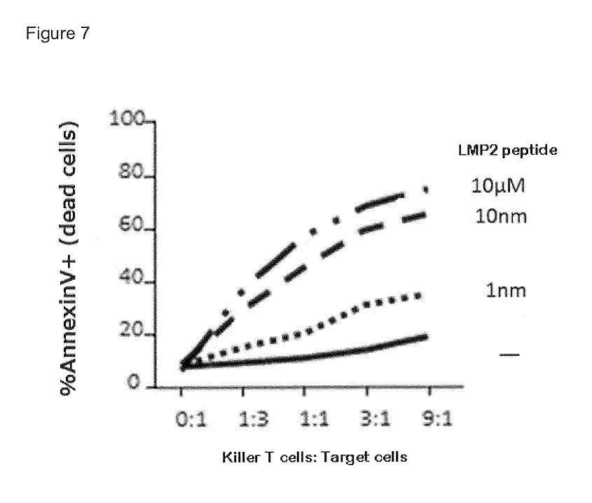

THP1 leukemia cells that were used as target cells were labelled with CFSE. The above obtained T cells (killer T cells) and the CFSE-labelled THP1 cells were added to each well of a 96-well round bottom plate at different effector/target cell ratios of 0:1, 1:3, 1:1, 3:1 and 9:1. The cells were incubated in the presence or absence of the peptide for 4 hours. The ratio of Annexin V positive cells to PI (Propidium Iodide) positive cells among the CFSE positive sorted cells were determined to confirm death rate of the target cells. Results are shown in FIG. 7.

Thus prepared killer T cells killed only little target cells when absence of the LMP2 peptide. In contrast, those killer T cells effectively killed the target cells in response to the amount of the LMP2 peptide. The killer activity was comparative to the original killer cells from which the T-iPS cells were established (FIG. 5). It could be concluded that the CTL activated in the thymus from T cell progenitors derived from T-iPS cells could provide considerably strong antigen specific cytotoxic activity.

In view of the result of Example C, T cell progenitors derived from T-iPS cells differentiate in the thymus where HLA molecules present and activated when stimulated by the antigen to give antigen specific CTLs.

1. A method for generating T cell progenitors for immune cell therapy, which comprises the steps of differentiating pluripotent stem cells bearing rearranged T cell receptor genes into T cell progenitors in vitro.

2. The method according to claim 1, wherein the T cell progenitors are selected from the group consisting of CD34⁺CD5⁺CD4⁻CD8⁻ cells, CD34⁺CD38⁻CD45RA⁻CD10⁻ cells, CD45RA⁺CD10⁺CD7⁻CD5⁻ cells, CD45RA⁺CD10⁺CD7⁺CD5⁻ cells, CD45RA⁺CD10⁺CD7⁺CD5⁺ cells, CD3⁻CD4⁺CD8⁻ cells and CD4⁺CD8⁺ cells.

3. The method according to claim 1, wherein the pluripotent stem cells bearing rearranged T cell receptor genes are iPS cells induced from a human cytotoxic T cell.

4. The method according to claim 3, wherein the human cytotoxic T cell is induced from human peripheral mononuclear cells (PBMCs) by stimulating the PBMCs with an antigen.

5. The method according to claim 4, wherein the antigen is a cancer antigen and the immune cell therapy is for the treatment of a cancer patient.

6. A method for immune cell therapy, which comprises generating T cell progenitors from pluripotent stem cells bearing rearranged T cell receptor genes and transferring the T cell progenitors to a patient in need of the treatment.

7. The method according to claim 6, wherein the T cell progenitors are selected from the group consisting of CD34⁺CD5⁺CD4⁻CD8⁻ cells, CD34⁺CD38⁻CD45RA⁻CD10⁻ cells, CD45RA⁺CD10⁺CD7⁻CD5⁻ cells, CD45RA⁺CD10⁺CD7⁺CD5⁻ cells, CD45RA⁺CD10⁺CD7⁺CD5⁺ cells, CD3⁻CD4⁺CD8⁻ cells and CD4⁺CD8⁺ cells.

8. The method according to claim 6, wherein the pluripotent stem cells bearing rearranged T cell receptor genes are iPS cells induced from a human cytotoxic T cell.

9. The method according to claim 8, wherein the human cytotoxic T cell is induced from human peripheral mononuclear cells (PBMCs) by stimulating the PBMCs with an antigen.

10. The method according to claim 9, wherein the antigen is a cancer antigen and the patient to be treated is a patient suffered from cancer.

11. The method according to claim 7, wherein the pluripotent stem cells bearing rearranged T cell receptor genes are iPS cells induced from a human cytotoxic T cell.

12. The method according to claim 11, wherein the human cytotoxic T cell is induced from human peripheral mononuclear cells (PBMCs) by stimulating the PBMCs with an antigen.

13. The method according to claim 12, wherein the antigen is a cancer antigen and the patient to be treated is a patient suffered from cancer.
